(19) United States
(12) Patent Application Publication
(10) Pub. No.: US 2010/0111867 A1
(43) Pub. Date: May 6, 2010

(54) CARBORANE-PHOSPHONIUM COMPOUNDS AND THEIR USE IN BORON NEUTRON CAPTURE THERAPY AND IMAGING

(75) Inventors: Louis M. Rendina, New South Wales (AU); Joseph A. Ioppolo, New South Wales (AU)

Correspondence Address: Joseph R. Baker, APC;Gavrilovich, Dodd & Lindsey LLP, 4660 La Jolla Village Drive, Suite 750, San Diego, CA, 92122, US

(73) Assignee: THE UNIVERSITY OF SYDNEY, New South Wales (AU)

(21) Appl. No.: 12/526,844

(22) PCT Filed: Feb. 15, 2008
(86) PCT No.: PCT/AU08/00198

§ 371 Date: Aug. 12, 2009

(30) Foreign Application Priority Data
Feb. 16, 2007 (AU) 2007900804

(51) Int. Cl. A61K 49/00; C07F 5/02; A61K 31/69; A61P 35/00

(52) U.S. Cl. 424/9.1; 568/2; 514/64

(57) ABSTRACT

The invention describes compounds for use in BCNT. These compounds comprise a carborane group coupled to a phosphorus containing group. The compounds may comprise a carborane group coupled to a phosphonium group.

### TECHNICAL FIELD

The present invention relates to boronated phosphine derivatives and methods for making them. The present invention also relates to boronated phosphonium salts and their use as boron neutron capture therapy agents and imaging agents for tumors.

### BACKGROUND OF THE INVENTION

Delocalised lipophilic cations (DLCs) can readily traverse the lipophilic mitochondrial membrane and accumulate in mitochondria. The selection of DLCs for use as tumour imaging and anti-cancer agents has been based solely on their advantageous physical properties, lipophilicity and delocalised positive charge. A high negative electrical potential which exists across the mitochondrial membrane enhances the uptake of DLCs into the mitochondria. The potential difference across the membrane in normal, healthy cells has been determined to be −104 mV. Mitochondria from human colon carcinoma cells, for example, have been shown to consistently exhibit elevated membrane potentials of −163 mV, sufficient to account for a ten-fold increase of dye concentration in cancerous cells over healthy cells (J. S. Modica-Napolitano, J. R. Aprille, Adv. Drug Deliver. Rev. 2001, 49, 63).

Phosphonium salts represent a promising class of DLCs for use in tumour targeting and imaging. Tetraphenylphosphonium (TPP) salts, triphenylmethylphosphonium (TPMP) salts and related compounds based on these structures have demonstrated highly cancer-selective accumulation and cytotoxicity. TPP salts have been shown to accumulate selectively in the mitochondria of tumours, and radiolabelled TPP has been proposed as a potential agent for imaging tumours. For example, its tumour:healthy tissue ratio has been measured to be 30:1 in glioma (S. Davis, M. J. Weiss, J. R. Wong, T. J. Lampidis, L. B. Chen, J. Biol. Chem. 1985, 260, 13844). It also exhibits high uptake and retention in neurofibrosarcoma, another tumour of the central nervous system (CNS). This is significant as many CNS tumours are resistant to current cancer therapies. TPMP iodide, radio-labelled with the ¹¹C isotope, has been investigated for in vitro uptake and retention in canine brain tumour in the interest of imaging tumours using positron emission tomography (PET). Not only was this compound rapidly taken up and retained in the tumour, but it was retained for a significant time (ca. 95 min) and achieved a tumour:healthy tissue ratio of 48:1. This ratio is also one order of magnitude higher than for other available glioma PET tracers and for agents currently used for Boron Neutron Capture Therapy (BNCT). TPMP is also known to suppress tumour growth.

In the context of BNCT, incorporation of boron into this compound would potentially result in a selective delivery of boron to the tumour site. Furthermore, animal studies have demonstrated that the expected toxic effects of TPMP may not prevent its use in humans. Use of carboranes in BNCT is considered to be of benefit, due to the high number of boron atoms in the molecules. This reduces the drug dosage and irradiation time required in order to maximise tumour cell kill.

Agents that are selective toward certain cancers by targeting the mitchondria represent a new class of BNCT agents. The only example of a boron-containing DLC reported to date appears to be the 1,12-carborane analogue of dequalinium, which was found to accumulate selectively in human epidermoid carcinoma of the oral cavity and rat glioma in vitro. It also exhibited similar uptake and retention properties to Rhodamine 123, MKT-077 and TPP chloride, suggesting this and other boron-containing DLCs such as the phosphonium salts are worthy of investigation as potential BNCT agents.

In the mid 1980s, a 1,2-bis(diphenylphosphino)ethane (dppe) complex of gold(I) (see below) was prepared and exhibited higher cytotoxic and antitumour activity against a wide spectrum of tumours in vivo. This complex was believed to work by targeting the mitochondria, as a result of its balance between lipophilic and hydrophilic characteristics. This complex, due to the ligand's bidentate nature, is more stable with respect to ligand exchange reactions and less reactive toward thiols than monodentate phosphines.

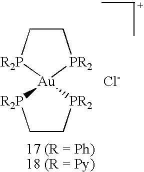

As these gold(I) compounds are cancer specific, derivatives incorporating boron-containing phosphine ligands may act as tumour-selective agents for BNCT. They could deliver boron as well as achieve a cytotoxic effect. Furthermore, the 100% abundant ¹⁹⁷Au nucleus has a significant thermal neutron cross-section (99 barns). The resulting ¹⁹⁸Au nucleus is unstable and emits a beta particle and a gamma ray. It also has a thermal neutron capture cross-section of 2.5×10⁴ barns and can absorb a further neutron, giving off another gamma ray. These gamma emissions are ionising and would contribute to the overall cytotoxic effect. Hence the synthesis of boron-containing analogues may have great potential for use in BNCT.

There is a need for boron-containing compounds suitable for BNCT and/or imaging applications.

### OBJECT OF THE INVENTION

It is the object of the present invention to at least partially satisfy the above need.

### SUMMARY OF THE INVENTION

In a broad form the present invention provides a compound comprising a carborane group coupled to a phosphorus containing group. The phosphorus containing group may bear a positive charge. It may be a cationic group. It may for example comprise a phosphonium group, or it may comprise a phosphine group coupled to (e.g. complexed with) a cation. The cation may comprise a metal. The metal may be in the +1 or +2 state. The metal may for example be gold, e.g. Au(I). Thus the phosphorus containing group may be a gold species. It may comprise a phosphine group complexed to Au(I). The compound may be suitable for use in BNCT. The compound may be water soluble. It may be soluble in an aqueous solvent.

In a first aspect of the invention there is provided a compound comprising a carborane group coupled to a phosphonium group. The compound may be a cytotoxic compound. The compound may be adapted to be used for Boron Neutron Capture Therapy and/or as a therapeutic agent for treatment and/or imaging of tumours, particularly brain tumours such as glioblastoma. It may be so adapted by virtue of the large number of boron atoms per molecule. It may be so adapted by virtue of having a phosphonium group coupled to the carborane group. It may be so adapted by virtue of comprising a delocalised lipophilic cation which can readily traverse a lipophilic mitochondrial membrane and accumulate in mitochondria.

The phosphonium group may be bonded directly to the carborane group. It may be bonded directly to a carbon atom of the carborane group. The phosphonium group may be an alkyldiaryl phosphonium group. It may be for example diphenylmethyl phosphonium. There may be more than one, e.g. 2, phosphonium groups coupled (e.g. bonded directly) to the carborane group. Each may be as described above.

In one embodiment the carborane group is a closo-carborane. In this case, the compound comprises a counterion to the phosphonium group. The counterion may be a halide, for example chloride, bromide or iodide. It may be a monovalent cation. It may be a divalent cation. It may be a trivalent cation. The closo-carborane group may be a dicarba-closo-dodecaborane group. It may be a 1,2-, 1,7- or 1,12-closo-carborane, i.e. the two carbon atoms of the dicarbacarborane may be in the 1 and 2 positions, 1 and 7 positions or 1 and 12 positions. The compound may be a 1,2-, 1,7- or 1,12-dicarba-closo-dodecaborane-alkyldiarylphosphonium halide. The compound may be 1,2-, 1,7- or 1,12-dicarba-closo-dodecaborane-diphenylmethylphosphonium iodide.

In another embodiment the carborane is a nido-carborane bearing a negative charge. In this case the compound is a zwitterion. In the zwitterion, the phosphonium group bears a positive charge and the carborane group bears a negative charge. The nido-carborane may be a dicarba-nido-undecaborane. It may be an alkyldiarylphosphonium-7,8- or 7,9-dicarba-nido-undecaborane. It may be diphenylmethylphosphonium-7,8-dicarba-nido-undecaborane or diphenylmethylphosphonium-7,9-dicarba-nido-undecaborane.

In a second aspect of the invention there is provided a process for making a compound according to the first aspect, said process comprising alkylating a precursor which comprises a closo-carborane group coupled to a phosphine group.

The step of alkylating may comprise reacting the precursor compound with an alkylating reagent. The alkylating reagent may comprise an alkyl group having a leaving group bonded thereto. The leaving group may be a halide, a tosylate, a triflate or some other suitable leaving group. Thus the alkylating agent may be an alkyl tosylate or an alkyl halide, for example an alkyl chloride, bromide or iodide. The process may also comprise making the precursor. The step of making the precursor may comprise reacting an anion of a closo-carborane with a halophosphine. The halophosphine may be a chlorophosphine e.g. chlorodiphenylphosphine. The anion may be made by reacting a closo-carborane with a base of sufficient strength to remove a hydrogen from the carborane. The closo-carborane may be 1,2-, 1,7- or 1,12-dicarba-closo-dodecaborane.

In one embodiment there is provided a process for making a zwitterionic compound according to the first aspect, said zwitterionic compound comprising a negatively charged carborane group coupled to a phosphonium group, said process comprising the steps of:
a) alkylating a precursor, said precursor comprising a closo-carborane group coupled to a phosphine group to form a product comprising a closo-carborane group coupled to a phosphonium group; and
b) deboronating the product obtained in step a).

In this embodiment step b) may comprise exposing the product obtained in step a) to a polar solvent. The polar solvent may be a polar organic solvent. It may comprise dimethyl sulfoxide or ethanol or water or N,N-dimethylformamide, for example. It may comprise a solution of fluoride ion in ethanol.

In another embodiment there is provided a process for making a compound according to the first aspect, said process comprising reacting a precursor which comprises a 1,2-, 1,7- or 1,12-dicarba-closo-dodecaborane group coupled to a diphenylmethylphosphine group with methyl iodide.

In another embodiment there is provided a process for making a compound according to the first aspect, said process comprising:
reacting 1,2-, 1,7- or 1,12-dicarba-closo-dodecaborane with a base of sufficient strength to deprotonate the dicarba-closo-dodecaborane to form an anion of the dicarba-closo-dodecaborane;
reacting the anion of the dicarba-closo-dodecaborane with a halodiarylphosphine to form a precursor; and
reacting the precursor with an alkyl iodide to form an alkyldiphenyl phosphonium iodide of 1,2-, 1,7- or 1,12-dicarba-closo-dodecaborane.

In another embodiment there is provided a process for making a compound according to the first aspect, said process comprising:
reacting 1,2-, 1,7- or 1,12-dicarba-closo-dodecaborane with a base of sufficient strength to deprotonate the dicarba-closo-dodecaborane to form an anion of the dicarba-closo-dodecaborane;
reacting the anion of the dicarba-closo-dodecaborane with a halodiarylphosphine to form a precursor;
reacting the precursor with an alkyl iodide to form an alkyldiphenylphosphonium iodide of 1,2-, 1,7- or 1,12-dicarba-closo-dodecaborane; and
deboronating the alkyldiphenylphosphonium iodide of 1,2-, 1,7- or 1,12-dicarba-closo-dodecaborane with fluoride in ethanol solution to generate the corresponding nido compound.

The invention also provides a compound made by the process of the invention. The compound may be adapted to be used for Boron Neutron Capture Therapy and/or as a therapeutic agent for treatment and/or imaging of tumours.

In a third aspect of the invention there is provided a composition comprising a compound according to the first aspect or a compound made by the process of the second invention, together with one or more clinically acceptable adjuvants and/or carriers. The composition may be suitable for use in Boron Neutron Capture Therapy and/or as a therapeutic agent for treatment and/or imaging of a tumour. It may be suitable for imaging tumours using PET. The tumour may be a brain tumour. It may be a glioblastoma.

In a fourth aspect of the invention there is provided a method for imaging and/or treating a tumour in a patient comprising the step of administering to said patient a compound according to the first aspect or a compound made by the process of the second invention or a composition according to the third aspect. The tumour may be a brain tumour. It may be a glioblastoma. The method (particularly in the case where the method is for imaging the tumour) may additionally comprise irradiating the tumour with neutrons during or after said step of administering. The irradiation may be sufficient to convert at least some of the 10B in said compound to ¹¹B. The process may comprise is allowing sufficient time following the administration for the compound to enter the tumour. It may comprise imaging the tumour, e.g. using PET, to determine whether the compound has entered the tumour. The administering may therefore be conducted when said imaging indicates that the compound has entered the tumour.

In an embodiment there is provided a method for imaging and/or treating a tumour in a patient comprising:
administering to said patient a compound according to the first aspect or a compound made by the process of the second invention or a composition according to the third aspect, and
irradiating the tumour with neutrons during or after said step of administering.

In another embodiment there is provided a method for imaging and/or treating a tumour in a patient comprising:
administering to said patient a compound according to the first aspect or a compound made by the process of the second invention or a composition according to the third aspect,
imaging said tumour, e.g. with PET, to determine whether the compound has entered the tumour, and
irradiating the tumour with neutrons when said imaging indicates that the compound has entered the tumour.

In a fifth aspect of the invention there is provided the use of a compound according to the first aspect or a compound made by the process of the second invention or a composition according to the third aspect for imaging and/or treating a tumour. The tumour may be a brain tumour. It may be a glioblastoma. There is also provided a s compound according to the first aspect or a compound made by the process of the second invention or a composition according to the third aspect when used for imaging and/or treating a tumour.

In a sixth aspect of the invention there is provided the use of a compound according to the first aspect or a compound made by the process of the second invention for the manufacture of a medicament for the treatment and/or imaging of a tumour. The tumour may be a brain tumour. It may be a glioblastoma.

In a seventh aspect of the invention there is provided a compound comprising a carborane group coupled to a phosphine group, said phosphine group being complexed to a gold species.

The gold species may comprise Au(I).

The compound may comprise a halide counterion.

The carborane group may be a 1,12-dicarba-closo-dodecaborane group. The carborane group may be coupled to two phosphine groups.

The compound may comprise two carborane groups, each coupled to a phosphine group, each of said phosphine groups being complexed to the same gold species so as to couple the two carborane groups to each other.

In an eighth aspect of the invention there is provided a process for making a compound comprising a carborane group coupled to a phosphine group, said phosphine group being complexed to a gold species, said process comprising exposing a precursor comprising a closo-carborane group coupled to a phosphine group to a reagent comprising gold.

The gold may comprise Au(I). The Au(I) may be [AuCl(SMe)₂].

The carborane group may be, or comprise, a 1,12-dicarba-closo-dodecaborane group.

In a ninth aspect of the invention there is provided a compound comprising a carborane group coupled to a phosphine group, said phosphine group being complexed to a gold species, said compound being made by the process of the eighth aspect.

In a tenth aspect of the invention there is provided a composition comprising a compound according to the seventh aspect or the ninth aspect together with one or more clinically acceptable adjuvants and/or carriers.

In an eleventh aspect of the invention there is provided a method for imaging and/or treating a tumour in a patient comprising the step of administering to said patient a compound according to the seventh or ninth aspect or a composition according to the tenth aspect. The tumour may be a brain tumour. It may be a glioblastoma.

In a twelfth aspect of the invention there is provided use of a compound according to the seventh or ninth aspect or a composition according to the tenth aspect for imaging to and/or treating a tumour. The tumour may be a brain tumour. It may be a glioblastoma.

In a thirteenth aspect of the invention there is provided use of a compound according to the seventh or ninth aspect for the manufacture of a medicament for the treatment and/or imaging of a tumour. The tumour may be a brain tumour. It may be a glioblastoma. The invention also provides use of a compound or a composition according to the invention for BNCT. It also provides use of a compound according to the invention for manufacture of a medicament for use in BNCT.

Compounds according to the invention include, but are not restricted to, (1,12-dicarba-closo-dodecaboranyl)diphenyl(1-bromo-2-(2-(2-ethoxyethoxy)ethoxy)ethyl)phosphonium bromide, μ-1,12-dicarba-closo-dodecaboranyl-1,12-bis(diphenylmethylphosphonium)iodide, bis(1-diphenylphosphino-1,12-dicarba-closo-dodecaborane)gold(I) chloride, chloro(1-diphenylphosphino-1,12-dicarba-closo-dodecaborane)gold(I), dichloro-μ-{1,12-bis(diphenylphosphino)-1,12-dicarba-closo-dodecaborane}digold(I), bis(1,12-bis(diphenylphosphino)-1,12-dicarba-closo-dodecaborane) gold(I) chloride, methyldiphenyl(1,12-dicarba-closo-dodecaboranyl)phosphonium iodide, methyldiphenyl(1,2-dicarba-closo-dodecaboranyl)phosphonium iodide, methyldiphenyl(1,7-dicarba-closo-dodecaboranyl)phosphonium iodide, methyldiphenyl(7,8-dicarba-nido-undecaboranyl)phosphonium and methyldiphenyl(7,9-dicarba-nido-undecaboranyl)phosphonium.

One of the advantages of the invention is that it provides a new class of potential boronated cancer treatment (BNCT) agents.

### DETAILED DESCRIPTION OF THE INVENTION

The present invention provides compounds which are suitable for use in Boron Neutron Capture Therapy, comprising a carborane group coupled to a phosphonium group or comprising a compound comprising a carborane group coupled to a phosphine group. The compounds may be cytotoxic, and may be toxic towards tumour cells. They may be non-toxic, or of low toxicity, towards tumour cells but be capable of decomposing in a beam of neutrons to produce products which are toxic towards tumour cells. They may be of use in treating and/or imaging tumours. They may be stable in air. They may be stable in water. They may be soluble (or at least partially soluble) in water. They may have low toxicity towards non-cancerous cells e.g. non-cancerous human cells. They may be non-pyrophoric.

Many carborane species seem to be stable (in the case of the monosubstituted 1,12-closo-carborane species) but are readily deboronated in polar solvents at room temperature in 1,2-, and 1,7-closo-carborane species. They are not particularly water-soluble but the carborane may be functionalised with a suitable group (e.g. functionalised with a glycerol group) to provide a water soluble substance.

The phosphonium group, or phosphine group, of the compounds of the present invention may be bonded directly to the carborane group. It may be coupled to the carborane group via a linker. It may be bonded or coupled to a carbon atom of the carborane group. The linker may be for example a hydrocarbon linker (e.g. aromatic, aliphatic, alicyclic, straight chain, branched, saturated, unsaturated, olefinic, acetylenic or some combination thereof) or it may comprise a suitable functional group (e.g. ester, ether, thioether, amine etc.). A PEG linker may be used for improved water-solubility. There may be more than one, e.g. 2, phosphonium groups, or phosphine groups, coupled (e.g. bonded directly) to the carborane group. Each of the phosphonium groups, or phosphine groups, may be as described herein. Each may be bonded or coupled to a carbon atom of the carborane group, in particular to a carbon atom of the carborane framework.

The phosphonium group may be an alkyldiaryl phosphonium group. Alternatively it may be an aryldialkyl phosphonium group or a trialkyl phosphonium group. The phosphonium group may have heteroaryl groups attached to the phosphorus atom. The alkyl and/or aryl and/or heteroaryl groups may optionally be substituted, e.g. with alkyl groups (for example C1-C6 straight chain alkyl, C2-C6 alkenyl or alkynyl, C3-C8 cycloalkyl, any of which may optionally be substituted), aryl groups (e.g. phenyl, naphthyl, anthracyl, phenanthryl, any of which may be optionally substituted), heteroaryl groups (e.g. pyridyl, furyl, pyranyl). Suitable phosphonium groups include diphenylmethyl phosphonium, trimethyl phosphonium, methyldiethyl phosphonium or ethyldimethyl phosphonium. All DLCs require at least one lipophilic (e.g. aromatic) group for biological activity.

The phosphine group may be a dialkyl phosphonium group, an aryl alkyl phosphonium group or a diaryl phosphonium group. The aryl and/or alkyl groups may be as described above (wherein the term aryl encompasses heteroaryl as described above).

The carborane group may be a closo-carborane group, i.e. a carborane group with a to closed cage structure. It may be an icosahedral carborane group. It may be a dicarba-carborane group. It may be a dicarba-closo-dodecaborane(12) group. It may have a cage structure of formula C₂B₁₀. The carborane group may have formula C₂B₁₀H. It may be a 1,2-, 1,7- or 1,12-closo-carborane group, i.e. the two carbon atoms of the dicarbacarborane group may be in the 1 and 2 positions, 1 and 7 positions or 1 and 12 positions. The phosphonium group may be attached to C1 or C2 of the carborane group. More than one phosphonium group may be present. The other carbon of the carborane group may be bonded to a hydrogen atom or to some other atom. It may be bonded to carbon. It may have an alkyl substituent e.g. methyl, ethyl, propyl, isopropyl or some other suitable substituent. It may have another phosphonium group. Almost any functional group is possible, provided that it is compatible with the carborane (e.g. strongly basic groups are not possible). Carboranes other than dicarba-docecaboranes or nido equivalents are possible. They may have more or less than two carbon atoms. They may have more or less total cage atoms. However, smaller carboranes (i.e. less boron atoms) are commonly quite unstable and/or water/air-sensitive. The [B₁₂H₁₂]²⁻ group is possible (i.e. no carbon atoms).

The carborane group may have no positive or negative electrical charge. In this case, the compound comprises a counterion in association with the phosphonium group which is coupled to the carborane group. The counterion may be a halide, for example chloride, bromide or iodide, or it may be some other counterion, e.g. acetate, nitrate, sulfate, phosphate, perchlorate. It may be a pharmaceutically or veterinarily acceptable counterion. The counterion may be monovalent, or may be di-, tri- or tetra-valent. Any counterion (other than hydroxide or fluoride) is possible. In the event that the counterion has a valency greater than 1, the resulting salt may comprise more than one carborane group. Thus for example, a salt comprising a carborane framework bearing a methyldiphenyl phosphonium group and a divalent anion may have a stoichiometry such that there are two carborane groups per anion. In the event that the carborane framework bears two methyldiphenyl phosphonium groups, a salt with a divalent anion may have a 1:1 stoichiometry.

A suitable 1,2-carborane is represented as:

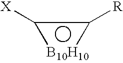

wherein X is the phosphonium group and R is the substituent on the other carbon atom.

The carborane group may be a nido-carborane group. A nido-carborane group is related to a closo-carborane group but is lacking one of the boron atoms of the cage structure. It is therefore an open cage structure. It may be obtained from a closo-carborane group by removal of a boron atom (i.e. by deboronation). The carborane group may be a dicarba-nido-carborane group. It may be a dicarba-nido-undecaborane group. It may have a cage structure of formula C₂B₉. It may have formula C₂B₉H (in particular C₂B₉H⁻). The hydrogen atom of the C₂B₉H may be associated with two different boron atoms of the carborane structure. The removed (or absent) boron atom may be a boron atom which, in the corresponding closo-carborane group, is adjacent to two different carbon atoms. The nido-carborane group bears a negative charge. Thus the compound is a zwitterion, comprising a phosphonium group having a positive charge and a carborane group having a negative charge. The compound may therefore have no counterion.

In compounds according to the present in which bromine is present, bromine may be replaced by ¹⁸F, which can then be imaged using positron emission tomography (PET). Bromine itself can also be imaged using synchrotron radiation induced X-ray emission (SRIXE). Thus the compounds of the present invention may comprise ¹⁸F, particularly when said compounds are to be used for imaging purposes.

The compounds of the present invention may be suited for treatment of brain tumours. They may be capable of crossing the blood-brain barrier. It is known that related compounds, e.g. triphenyl methyl phosphonium salts, may be capable of crossing the bloodd-brain barrier.

The compounds of the present invention may be pharmaceutically acceptable. They may be veterinarily acceptable. In some cases the compounds comprise one or more asymmetric centres. They may be optically active. They may have diastereomeric forms. In these cases, the present invention encompasses any and all of the optical isomers and/or diastereomers of the compound (either separately or mixed), as well as compositions comprising any or all of said optical isomers and/or diastereomers and use thereof as described herein. The process described for making the compounds may comprise the step of resolving the optical isomers and/or of separating the diastereomers.

The present invention also provides a process for making a compound according to the first aspect. The process comprising alkylating a precursor which comprises a closo-carborane group coupled to a phosphine group. Such precursors are known compounds, and may be made by known methods (e.g. C. Villas, R. Benakki, F. Teixidor, J. Casabo, Inorg. Chem., 1995, 34, 3844; W. E. Hill, L. M. Silva-Trivino, Inorg. Chem. 1979, 18, 361; N. N. Godovikov, V. P. Balema, E. G. Rys, Russ. Chem. Rev. 1997, 66, 1017), the contents of which are incorporated herein by reference. Thus, in a typical synthesis, an anion of a carborane is treated with a halophosphine in order to generate the corresponding phosphine substituted carborane. The anion of the carborane may be obtained by treatment of the corresponding carborane with a strong base, for example butyl lithium. This reaction is commonly conducted in a polar solvent capable of dissolving both reagents and product, and commonly is conducted at low temperature, e.g. less than about 0° C., or less than about −10, −20, −30, −40 or −50° C. It may be conducted at about −78° C., or at about −70, −60, −50, −40, −30, −20, −10, −5 or 0° C. The halophosphine may be a chlorophosphine or a bromophosphine or an iodophosphine. It may have two alkyl or two aryl or one aryl and one alkyl group attached to the phosphorus. One or more of the alkyl or aryl groups may be replaced by a heteroaryl group, and the alkyl, aryl and heteroaryl groups, if present, may optionally be substituted. A suitable phosphine is chlorodiphenyl phosphine. It will be readily apparent to one skilled in the art which phosphines are suitable for making a desired precursor, and which precursors are suitable for making a particular closo-carborane derivative.

The alkylation of the precursor may comprise reacting the precursor compound with an alkylating agent. The alkylating agent may be alkyl halide, for example an alkyl chloride, bromide or iodide, or may be an alkyl group having some other suitable leaving group. The alkyl group may be for example a C1-C6 straight chain alkyl, C2-C6 alkenyl or alkynyl, C3-C8 cycloalkyl, any of which may optionally be substituted. Suitable alkyl groups include methyl, ethyl, propyl, isopropyl, n-butyl, i-butyl, cyclopentyl etc. The alkylation may be conducted using an excess of the alkylating agent. Sometimes a very large excess (>100:1 molar ratio) is required (e.g. 120:1 or 150:1 or 125:1 or 110:1). The excess may be, on a molar basis, at least about 1.1, or at least about 1.2, 1.3, 1.4, 1.5, 2, 2.5, 3, 3.5, 4, 4.5 or 5 or even 100, or between about 1.1 and about 100, or between about 1.5 and 100, 2 and 5, 1.1 and 3, 1.1 and 2, 1.5 and 4 or 1.5 and 3, e.g. about 1.2, 1.3, 1.4, 1.5, 2, 2.5, 3, 3.5, 4, 4.5 or 5 or 100. The reaction may be carried out at room temperature or at elevated temperature, e.g. about 30, 40, 50, 60 or 70° C. It will be understood that in the event that the reaction is conducted above the boiling point of the alkylating agent, it may be necessary to conduct the reaction in a pressure vessel to avoid loss of the alkylating reagent.

The above alkylation reaction provides a phosphonium substituted carborane as described in the first aspect of the invention. The inventors have discovered, unexpectedly, that certain of such phosphonium substituted carboranes, can deboronate under neutral pH conditions to provide the corresponding nido-carborane phosphonium ylides in which the nido-carborane group has a negative charge and the phosphonium group bears a positive charge. The structure of the nido-carborane group has been described above, and is shown in the figures. The deboronation reaction is promoted by exposure of the compound to a polar solvent. The polar solvent may be protic (e.g. an alcohol) or aprotic. Suitable polar solvents include dimethyl sulfoxide, N,N-dimethylformamide, acetone, propylene carbonate, hexamethyl phosphoramide, ethanol or other alcohols (e.g. methanol, propanol, isopropanol) or mixtures thereof. The solvents may optionally be mixed with water or they may not be mixed with water. They may be anhydrous. They may have salts dissolved therein, e.g. a fluoride salt such as caesium fluoride. The deboronation reaction may be conducted at any suitable temperature, e.g. between about 5 and about 90° C., or between about 5 and 80, 5 and 50, 5 and 30, 5 and 20, 10 and 90, 20 and 90, 50 and 90, 20 and 50 or 20 and 30° C., e.g. about 5, 10, 15, 20, 25, 30, 40, 50, 60, 70, 80 or 90° C. It may be conducted at room temperature.

The present invention also provides a compound comprising a carborane group coupled to a phosphine group, said phosphine group being complexed to a gold species.

The gold species may comprise Au(I). It may have two groups complexed thereto. It may comprise —AuCl. It may comprise —Au⁺—. The compound may comprise a counterion. The counterion may be a halide counterion, e.g. chloride, bromide or iodide. It may be a monovalent counterion. It may be a di- or trivalent counterion.

The carborane group may be as described earlier. It may be for example a 1,12-dicarba-closo-dodecaborane group. The carborane group may be coupled to two phosphine groups. In this event, each of the phosphine groups may be coupled to a carbon atom of the carborane group.

The compound may comprise two carborane groups, each coupled to a phosphine group, each of said phosphine groups being complexed to the same gold species so as to couple the two carborane groups to each other. Thus the compound may be of structure:

carborane group-phosphine group-gold species-phosphine group-carborane group in which the carborane groups may be the same or may be different, and the phosphine groups may be the same or may be different. The compound may comprise a counterion, e.g. a halide ion. Each of the carborane groups, independently, may comprise a substituent, e.g. a phosphine group, which is not complexed to the gold species. These may be coupled, e.g. bonded, to a carbon atom of the carborane framework.

The invention also provides a process for making a compound comprising a carborane group coupled to a phosphine group, said phosphine group being complexed to a gold species. The process comprising exposing a precursor comprising a closo-carborane group coupled to a phosphine group to a reagent comprising gold. The gold may comprise Au(I). The reagent may comprise an Au(I) complex. It may comprise an Au(I) halide complex. It may comprise an Au(I) chloride complex. It may comprise an Au(I)Cl complex of a thiol. The thiol may be an alkyl thiol. The alkyl group may be a C1 to C6 alkyl group. The alkyl group may be linear, or may be branched (for C3 to C6) or may be cyclic (for C3 to C6). It may be unsaturated. It may be optionally substituted. The reagent may be for example [AuCl(SMe)₂].

The stoichiometry of the reaction may be such that the desired product is obtained. Thus for example if a compound containing a phosphine coupled to AuCl is desired, a 1:1 molar ratio of precursor to reagent may be used, whereas if a compound containing two phosphines coupled to Au(I) (e.g. carborane group-phosphine group-gold species-phosphine group-carborane group) is desired, a 2:1 molar ratio may be used.

The reaction commonly comprises simply dissolving the reagent and precursor in the desired ratio in a suitable solvent, e.g. dichloromethane. The reaction may be conducted at room temperature or some other suitable temperature (e.g. about 15, 20, 25, 30, 35 or 40° C.). The reaction time may be between about 1 and 24 hours or more, or 1 to 12, 1 to 6, 6 to 24, 12 to 24 or 12 to 18 hours, e.g. about 1, 2, 3, 4, 5, 6, 9, 12, 15, 18, 21 or 24 hours. The reaction may optionally be conducted under in inert atmosphere, e.g. under nitrogen, carbon dioxide, helium, argon etc. The reaction mixture may be agitated, e.g. stirred or shaken, during the reaction, or may not be agitated.

The invention also provides a composition for use in Boron Neutron Capture Therapy comprising a compound according to the invention or a compound made by the process of the invention, together with one or more clinically acceptable adjuvants and/or carriers. A person skilled in the art will appreciate that compositions embodying the present invention may be used, for example for imaging or treating a tumour. Use of different boron compounds to those disclosed herein in such applications has been discussed in the literature (e.g. J. D. Steichen, M. J. Weiss, D. R. Elmaleh, R. L. Martuza, J. Neurosurg. 1991, 74, 116; I. Madar, J. H. Anderson, Z. Szabo, U. Scheffel, P. Kao, H. T. Ravert, R. F. Dannals, J. Nuc. Med., 1999, 40, 1180; K. Yokoyama, S. Miyatake, Y. Kajimoto, S. Kawabata, A. Doi, T. Yoshida, T. Asano, M. Kirihata, K. Ono, T. Kuroiwa, J. Neuro-Oncol., 2006, 78, 227; R. F. Barth, J. A. Coderre, M. G. H. Vicente, T. E. Blue, Clin. Cancer Res., 2005, 11, 3987; A. H. Soloway, W. Tjarks, B. A. Barnum, F-G. Rong, R. F. Barth, I. M. Codogni, J. G. Wilson, Chem. Rev. 1998, 98, 1515; J. Patel, D. Rideout, M. R. McCarthy, T. Calogeropoulou, K. S. Wadwa, A. R. Oseroff, Anticancer Res., 1994, 14, 21). In the imaging and therapy applications of the present invention, the compositions embodying the present invention are used in place of the boron compounds used by earlier work in the field.

Boron neutron capture therapy (BNCT) is a binary therapy for the treatment of cancer. It is currently being used clinically for treated advanced glioma. The two components of this binary therapy are a ¹⁰B-containing drug and thermal neutrons. The drug is administered into the body and allowed time to distribute and accumulate in the cancerous tissue. Beams of thermal neutrons are then directed at the cancer site. When a ¹⁰B nucleide absorbs a thermal neutron it readily fissures into two charged, high-energy particles—an alpha particle (He²⁺) and a lithium ion (Li³⁺). These ions destroy the cancerous cells to which the ¹⁰B-containing agent and thermal neutrons have been localised. (A. H. Soloway, W. Tjarks, B. A. Barnum, F-G. Rong, R. F. Barth, I. M. Codogni, J. G. Wilson, Chem. Rev. 1998, 98, 1515)

The uptake and distribution of delocalised lipophilic cations (DLCs) in vitro and in vivo has been investigated using radio-labelling. The uptake and retention of tetraphenylphosphonium (TPP) chloride radio-labelled with ³H has been reported. (J. D. Steichen, M. J. Weiss, D. R. Elmaleh, R. L. Martuza, J. Neurosurg. 1991, 74, 116). The uptake and retention of triphenylmethylphosphonium (TPMP) iodide radio-labelled with ¹¹C has been reported using PET. In this case the tumour was imaged within the brain of a canine model. (I. Madar, J. H. Anderson, Z. Szabo, U. Scheffel, P. Kao, H. T. Ravert, R. F. Dannals, J. Nuc. Med., 1999, 40, 1180). The inventors believe that at present the only known DLC containing carborane is a 1,12-dicarba-closo-dodecaborane derivative of dequalinium named dequalinium-B. This has been studied for uptake in human epidermoid carcinoma of the oral cavity and rat glioma in vitro. (D. M. Adams, W. Ji, R. R. Barth, W. Tjarks, Anticancer Res. 2000, 20, 3395)

In BNCT, two separate components which individually have only minor effects on cells, are brought together in or near a tumour so as to interact in order to damage the tumour cells. The first of these components is ¹⁰B, which may be incorporated as described herein into a compound which can accumulate in a tumour cell. The second component is low energy neutrons. Exposure of the ¹⁰B atoms to the low energy neutrons can generate high energy transient ¹¹B atoms, which can disintegrate to generate alpha particles, ⁷Li recoil nuclei. Tumour cells may be selectively killed by the alpha particles and ⁷Li fission products. This may specifically target the tumour cells in which the boron compound is concentrated and leave adjacent normal cells largely unharmed. BNCT may be particularly suited for treatment of brain tumours, particularly highly malignant brain tumours such as glioblastoma multiforme

Thus, following administration of the boron compound to the patient and subsequent accumulation of the boron compound in the tumour cells, an epithermal beam of neutrons is directed towards the tumour. As the neutrons pass through the tissue they rapidly lose energy by elastic scattering until they end up as thermal neutrons. The thermal neutrons interact with the ¹⁰B atoms to generate ¹¹B atoms as described above, leading to killing of the tumour cells.

In imaging applications, the compound, or composition, of the present invention may be administered to a patient prior to the imaging step. Thus the compound may be administered to the patient, and sufficient time may then be allowed after the commencement of the administration (optionally after the completion of the administration) for the compound to reach sufficient concentration in the target location of the body of the patient for imaging. The target location may be a tumour, e.g. a brain tumour. It may be a glioblastoma. The imaging is then conducted. The imaging may comprise PET imaging or some other suitable imaging technique. It may be an imaging technique capable of imaging the compound administered. The compounds may therefore be used for imaging tumours, e.g. glioblastomas, in a patient.

The patient may be a human patient, or may be a non-human patient. The patient may be a mammal, e.g. a dog, a cat, a cow, a horse or some other mammal.

The compounds of the present invention may be administered as compositions either therapeutically or for imaging purposes. In a therapeutic application, compositions are administered to a patient already suffering from a disease, in an amount sufficient to cure or at least partially arrest the disease and its complications. The composition should provide a quantity of the compound or agent sufficient to effectively treat the patient.

The therapeutically effective dose level for any particular patient will depend upon a variety of factors including: the disorder being treated and the severity of the disorder; activity of the compound or agent employed; the composition employed; the age, body weight, general health, sex and diet of the patient; the time of administration; the route of administration; the rate of sequestration of the agent or compound; the duration of the treatment; drugs used in combination or coincidental with the treatment, together with other related factors well known in medicine.

One skilled in the art would be able, by routine experimentation, to determine an effective, non-toxic amount of agent or compound which would be required to treat applicable diseases.

Generally, an effective dosage is expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Typically, in therapeutic applications, the treatment would be for the duration of the disease state.

Further, it will be apparent to one of ordinary skill in the art that the optimal quantity and spacing of individual dosages will be determined by the nature and extent of the disease state being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the composition given per day for a defined number of days, can be ascertained by those skilled in the art using conventional course of treatment determination tests.

In general, suitable compositions may be prepared according to methods which are known to those of ordinary skill in the art and accordingly may include a pharmaceutically acceptable carrier, diluent and/or adjuvant.

These compositions can be administered by standard routes. In general, the compositions may be administered by the parenteral (e.g., intravenous, intraspinal, subcutaneous or intramuscular), oral or topical route. More preferably administration is by the parenteral route.

The carriers, diluents and adjuvants must be “acceptable” in terms of being compatible with the other ingredients of the composition, and not deleterious to the recipient thereof.

Examples of pharmaceutically acceptable carriers or diluents are demineralised or distilled water; saline solution; vegetable based oils such as peanut oil, safflower oil, olive oil, cottonseed oil, maize oil, sesame oils, arachis oil or coconut oil; silicone oils, including polysiloxanes, such as methyl polysiloxane, phenyl polysiloxane and methylphenyl polysiloxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

The compositions of the invention may be in a form suitable for administration by injection, in the form of a formulation suitable for oral ingestion (such as capsules, tablets, caplets, elixirs, for example), in an aerosol form suitable for administration by inhalation, such as by intranasal inhalation or oral inhalation, in a form suitable for parenteral administration, that is, subcutaneous, intramuscular or intravenous injection.

For administration as an injectable solution or suspension, non-toxic parenterally acceptable diluents or carriers can include water (e.g. BP or USP), Ringer's solution, isotonic saline, phosphate buffered saline, ethanol and 1,2 propylene glycol.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include emollients, emulsifiers, thickening agents, preservatives; bactericides and buffering agents.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

The emulsions for oral administration may further comprise one or more emulsifying agents. Suitable emulsifying agents include dispersing agents as exemplified above or natural gums such as guar gum, gum acacia or gum tragacanth.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which is incorporated herein by reference.

### BRIEF DESCRIPTION OF THE DRAWINGS

A preferred embodiment of the present invention will now be described, by way of an example only, with reference to the accompanying drawings wherein:

FIG. 1 is a scheme showing synthesis of 1,2-dicarba-closo-dodecaborane-diphenylmethylphosphonium iodide and its deboronation (reagents: (i) MeL (ii) CsF, EtOH);

FIG. 2 is a scheme showing synthesis of 1,7-dicarba-closo-dodecaborane-diphenylmethylphosphonium iodide and its deboronation (reagents: (i) MeL (ii) CsF, EtOH);

FIG. 3 is a scheme showing synthesis of 1,12-dicarba-closo-dodecaborane-diphenylmethylphosphonium iodide (reagents: (i) MeL (ii) CsF, EtOH);

FIG. 4 shows an ORTEP representation and atomic numbering scheme of compound 7;

FIG. 5 is a scheme showing synthesis of a water-soluble bromoether phosphonium salt;

FIG. 6 is a scheme showing synthesis of diphosphonium compound and degradation in ethanol;

FIG. 7 is a scheme showing synthesis of gold(I) complexes of carboranyl phosphine 21; and

FIG. 8 is a scheme showing Synthesis of gold(I) complexes of carboranyl phosphine 11.

### DETAILED DESCRIPTION OF THE PREFERRED EMBODIMENTS

The first examples of arylphosphonium salts containing a dicarba-closo-dodecaborane(12) (closo-carborane) are reported herein. In contrast to the 1,12-carborane derivative, the 1,2- and 1,7-isomers undergo a deboronation reaction in polar solvents to afford the corresponding nido-carborane phosphonium zwitterions.

The present specification describes the synthesis and characterisation of novel carborane-containing phosphonium derivatives. TPMP analogues that have one phenyl group replaced by either a closo-1,2-, 1,7- or 1,12-carborane are disclosed. In two out of three cases, a deboronation reaction occurs in polar solvents to afford the corresponding nido-carborane phosphonium zwitterions.

Syntheses of trisubstituted phosphines from the parent closo-carboranes were based upon the known preparation of the closo-1,2-carborane derivative 1. One equivalent of n-BuLi was treated with closo-1,2-, 1,7- or 1,12-carborane in (DME) (or THF in the case of 1,7- and 1,12-carboranes) at low temperatures to minimise disproportionation and hence disubstitution at the carbon atoms, and chlorodiphenylphosphine was then added to afford the desired phosphine compounds 1-3. Small amounts of the disubstituted carborane derivative were also obtained in all three cases and could be separated readily from the monosubstituted species by means of column chromatography on silica.

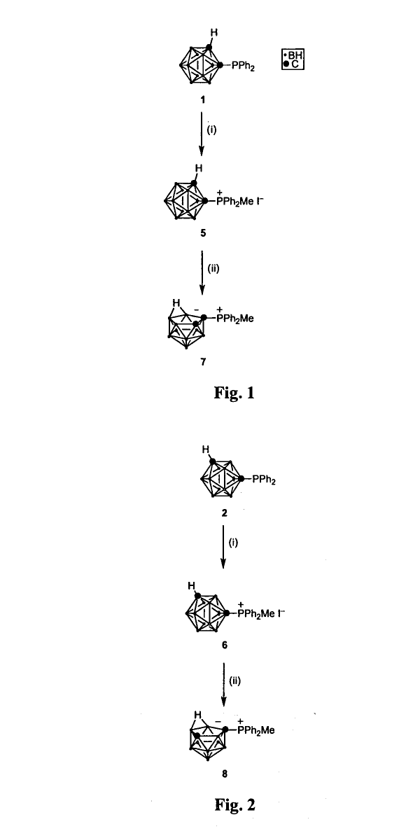

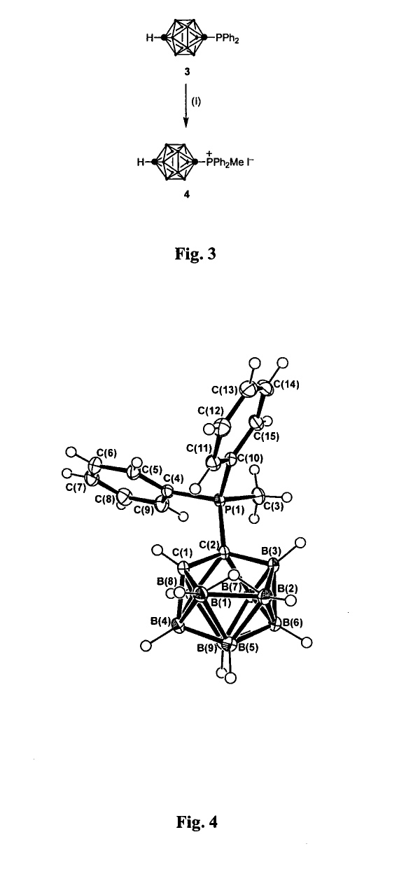

The synthesis of the phosphonium salts was achieved in all cases by a facile methylation reaction between 1-3 and MeI to afford the corresponding phosphonium iodides 4-6 (FIGS. 1-3). An excess of MeI was used in the reaction as this step was generally not high-yielding. The inventors hypothesise that this is because the carborane cage is somewhat electron-withdrawing thereby deactivating the phosphorus centre toward electrophiles. The characterisation of 4-6 was primarily achieved by means of mass spectrometry and NMR spectroscopy. The Me group exhibited a downfield-shifted doublet in the ¹H NMR spectra, with ²J_{PH}=13-14 Hz. A single peak in the ³¹P{¹H} NMR spectra at ca. δ 30 was located downfield from the peak of the corresponding tertiary phosphine precursor.

Compounds 5 and 6 were found to spontaneously deboronate to afford the corresponding nido-carborane zwitterions 7 and 8, respectively, in polar solvents such as is DMSO; reaction with fluoride ion in EtOH solution resulted in the rapid formation of the pure zwitterionic products with optimised yields (FIGS. 1 and 2). The deboronation reaction was not observed for 4, which contains 1,12-carborane. The inventors hypothesise that 4 is highly resistant to the deboronation reaction because no boron atoms within the cluster are bonded to two adjacent and electronegative carbon atoms. The facile deboronation of closo-1,2-carborane under mild, non-basic conditions is known with alcohols, water and acids (E. Svantesson, J. Pettersson, Å. Olin, K. Markides, S. Sjöberg, Acta Chem. Scand., 1999, 53, 731. V. A. Ol'shevskaya, R. Ayuob, Z. G. Brechko, P. V. Petrovskii, E. G. Kononova, G. L. Levit, V. P. Krasnov, V. N. Charushin, O. N. Chupakhin, V. N. Kalinin, J. Organomet. Chem., 2005, 690, 2761. Y.-J. Lee, J.-D. Lee, J. Ko, S.-H. Kim, S. O. Kang, Chem. Commun., 2003, 1364) and has also been observed when functional groups located α- to the cage are electron-withdrawing and the compounds are exposed to polar solvents containing small amounts of H₂O or MeOH (J. J. Schaeck, S. B. Kahl, Inorg. Chem., 1999, 38, 204). In the present case, the deboronation reactions occur in polar solutions such as dry DMF and thus most likely proceed by nucleophilic attack of the iodide counter-ion on the most electrophilic boron atoms in the cage, i.e. B3 (or B6) for 1,2-carborane and B2 (or B3) for 1,7-carborane. Interestingly, the deboronation reaction is not reported for boron-substituted alkylphosphonium bromide salts of closo-1,2-carborane in which the cationic centre is separated from the carborane cage by at least four bonds (W. Chen, M. Diaz, J. J. Rockwell, C. B. Knobler, M. F. Hawthorne, C. R. Acad. Sci. Paris, Série IIc, 2000, 3, 223).

Degradation of the closo-carborane cage in 5 and 6 to the corresponding nido derivative was confirmed by the presence of an upfield broad peak at δ-2 in the ¹H{¹¹B} NMR spectra of 7 and 8 which is attributed to the bridging H-atom, as well as the significant upfield shifts of resonances in the ¹¹B{¹H} NMR spectra. In addition to the NMR studies, the structure of 7 was confirmed by X-ray crystallography.

Colourless prismatic crystals were grown from the slow diffusion of diethyl ether into a DMF solution containing 7 over several days. X-ray data: Formula C₁₅H₂₄B₉P, M 332.60, Monoclinic, space group P2₁/c(#14), a=11.4316(14), b=9.4232(10), c=17.547(2) Å, =102.805(6), V=1843.2(4) Å³, D_{c}=1.199 g cm⁻³, Z=4, crystal size=0.314 by 0.290 by 0.157 mm, colour colourless, habit prism, temperature=150(2) K, (MoK)=0.71073 Å, (MoK)=0.143 mm⁻¹, T(SADABS)_{min,max}=0.750, 0.98, 2ₘₐₓ=61.28, hkl range=−16 16, −13 13, −25 25, N=37976, N_{ind}=5637 (R_{merge} 0.0635), N_{obs}=4587 (I>2(I)), N_{ind}=242, residuals* R1(F)=0.0444, wR2(F²)=0.1318, GoF(all)=1.085, =−0.584, 0.499 e⁻Å⁻³.
*R1=∥Fₒ|−|Fₒ∥/|Fₒ| for Fₒ>2(Fₒ); wR2=(w(Fₒ²−Fₒ²)²/(wFₒ²)²)^{1/2} all reflections w=1/[²(Fₒ²)+(0.0632P)²+0.8298P], where P=(Fₒ²+2Fₒ²)/3.

An ORTEP representation of 7 is presented in FIG. 4, shown with 50% probability ellipsoids. Selected bond distances (Å) are: C(1)-C(2)=1.5680(17), C(2)-P(1)=1.7867(14), C(3)-P(1)=1.7957(14), C(4)-P(1)=1.7981(13), C(10)-P(1)=1.7982(13). Selected bond angles (degrees) are: C(1)-C(2)-P(1)=116.46(9), C(5)-C(4)-P(1)=120.96(10), C(9)-C(4)-P(1)=119.20(10), C(15)-C(10)-P(1)=119.80(10), C(11)-C(10)-P(1)=120.64(10), C(2)-P(1)-C(3)=109.76(6), C(2)-P(1)-C(4)=112.87(6), C(3)-P(1)-C(4)=108.23(6), C(2)-P(1)-C(10)=106.88(6), C(3)-P(1)-C(10)=109.27(6), C(4)-P(1)-C(10)=109.79(6). The bond angles and lengths fall in the range of typical alkyltriarylphosphonium salts, e.g. MePh₃P⁺X⁻(X═ClO₄, BF₄), and are almost identical to those found in the phosphonium salt MePh₃P⁺(7,8-Et₂-7,8-nido-C₂B₉H₁₀)⁻. The central phosphorus atom is close to an ideal tetrahedral geometry with bond angles ranging from 112.87(6)° (C(2)-P(1)-C(4)) to 106.88(6)° (C(2)-P(1)-C(10)). The C(2)-P(1) bond length of 7 (1.7867(14) Å) is only marginally shorter than the C(10)-P(1) and C(4)-P(1) bond lengths (1.7982(13) and 1.7981(13) Å, respectively) indicating there is very little double-bond character between the phosphorus centre and the cage carbon atom. Hence, a zwitterionic structure bearing separate but delocalised positive and negative charges at the MePh₂P moiety and nido-carborane cage, respectively, is consistent with the X-ray data rather than an ylide-like structure in which there exists significant double-bond character and, consequently, a shortened phosphorus-carbon bond. Such bond shortening has been observed in simple phosphorus ylide compounds, e.g. PPh₃CH₂ (1.661 Å) and phosphorus-borane ylides such as endo-7-[Ph₂(H)P]-8-R-hypho-7,8-C₂B₆H₁₁, in which the P—C bond lengths were found to be 1.737(3) and 1.742(3) Å for R═H and Me, respectively. The X-ray structure of 7 also reveals a number of ring-stacking interactions present throughout the crystal lattice. The zwitterionic molecules pack together forming infinite one-dimensional chains through offset face-to-face interactions which are illustrated by a C(7)-C(12) distance of 3.45 Å. These chains pack closely together with adjacent chains forming a two-dimensional sheet-like motif, which propagates in the be-plane and is held together by π-BH interactions between the delocalised carborane cages and the phenyl rings of adjacent molecules. Indicative BH-ring centroid distances are the B(5)H-ring centroid of the C(12) containing is ring=3.25 Å and B(4)H-ring centroid of the C(8) containing ring=3.41 Å.

In conclusion, the first examples of arylphosphonium salts containing a closo-carborane are disclosed herein. In contrast to the 1,12-carborane derivative 4, the 1,2- and 1,7-isomers undergo a deboronation reaction in polar solvents to afford the corresponding zwitterionic nido-carborane phosphonium species. Preliminary in vitro cytotoxicity screening of 4 against the SF268 (human glioblastoma) cell line demonstrated a favourable GI₅₀>40 μM compared with the control compound TPMP (GI₅₀=12.5 μM). This is ideal for BNCT as the drug would have limited effects on surrounding healthy tissue, and because required dose of the drug shall not be limited significantly by its inherent cytotoxicity.

### More Polar Carborane Phosphonium Salts

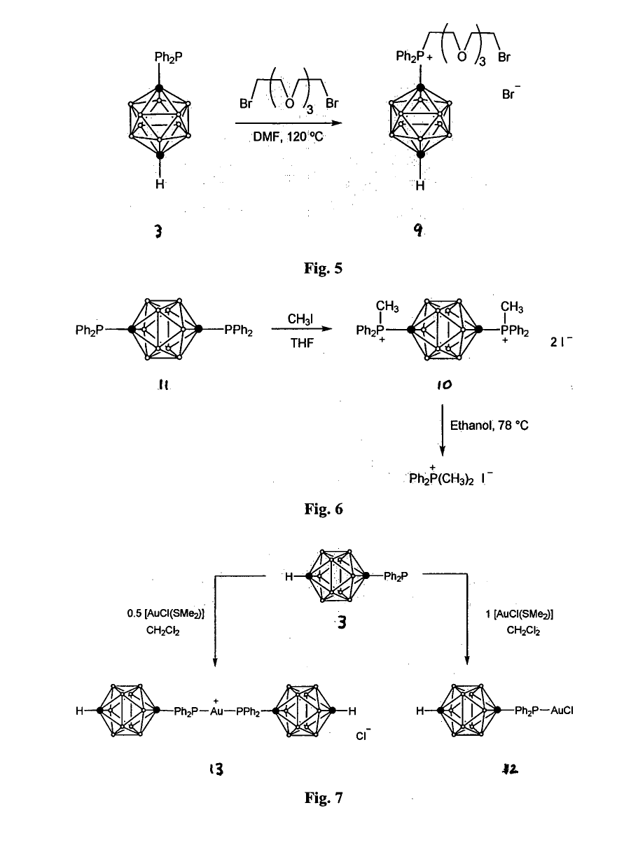

As outlined in FIG. 5, reaction between 3 and bis(2-(2-bromoethoxy)ethyl)ether produced the phosphonium salt 9. The reaction was carried out in d₇-dimethylformamide solvent at 140° C., and due to its water solubility, the product was able to be purified by extraction into water.

The synthesis of dication 1,12-carboranylene-1,12-bis(diphenylmethylphosphonium)iodide 10 was attempted. It was expected that this species would relatively water soluble as it contains two charges. The 1,12-carborane isomer was used so as to avoid deboronation, which, as the nido cage is monoanionic, would lead to a monocation overall. The disubstituted phosphine 11 was reacted with excess iodomethane as shown in FIG. 6. A precipitate formed which was later found to be the target compound, as evidenced by the ¹H, ³¹P{¹H} and ¹¹B{¹H} NMR spectra and elemental analysis. The mass spectrum of the crude reaction product contained an envelope of peaks centred at m/z=271.6 corresponding to the dication. It also contained a large envelope of peaks centred at m/z=343.8, which corresponds to the larger fragment if one C_{cage}—P bond were fissured in the spectrometer.

Attempts to alkylate phosphine 3 with alkyl bromides of structure Br—CH₂CH₂—X, where X is bromide or tosylate, failed to provide the desired bromoethyl phosphonium salt.

### Gold(I)-Phosphine Complexes.

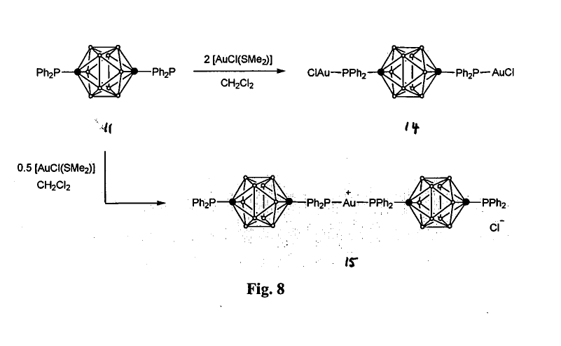

The first examples of gold(I)-phosphine complexes containing 1,12-dicarba-closo-dodecaborane(12) (1,12-carborane) are reported herein. Treatment of carborane phosphine 3 with the labile gold(I) precursor [AuCl(SMe₂)] (R. Uson, A. Laguna, M. Laguna, Inorg. Synth. 1989, 26, 85) in dichloromethane solution afforded the complex 12 or the salt 13 depending on the stoichiometry of the reaction (FIG. 7). Similarly, treating the same precursor with carborane bisphosphine 11 afforded either the mono- or di-nuclear species 14 or 15, respectively, depending upon the stoichiometry of the reaction (FIG. 8). All products were characterised primarily by means of multinuclear NMR spectroscopy, ESI-MS and microanalysis and in no case was deboronation of the carborane cage observed. In the case of complex 12, two distinct resonances were observed in the ¹¹B{¹H} NMR spectrum, consistent with only one of two carborane cage carbon atoms being functionalised by the phosphine. The ³¹P{¹H} NMR spectrum of 12 displays a singlet at δ 54.8. The FT-IR spectrum contains the characteristic ν(Au—Cl) stretch at 333 cm⁻¹ as has been previously observed with related gold(I) complexes. In a separate attempt the salt 13 was formed instead, displaying a molecular ion peak present at m/z 854.5 in the ESI mass spectrum. This is consistent with the proposed structure as shown. It is likely that the gold precursor had degraded before use and so less equivalents of gold(I) were added in this case. The ³¹P{¹H} NMR spectrum of 13 displays a singlet at δ0 41.9, which clearly indicates a different complex to 12.

For complexes 14 and 15, only singlets were observed in the ¹¹B{¹H} and ³¹P{¹H} NMR spectra consistent with the high symmetry associated with the carborane cage. The presence of a single (albeit broad) resonance in the ³¹P {¹H} NMR spectrum of 15 is most likely to be the result of a dynamic intramolecular fluxional process in which the two phosphorus environments become time-averaged on the NMR timescale. The FT-IR spectrum of 14 displayed a characteristic ν(Au—Cl) stretch at 334 cm⁻¹.

The first examples of gold(I)-phosphine complexes containing 1,12-carborane as part of the ligand framework were formed. In were the facile deboronation reactions observed that have been previously observed with analogous complexes containing the isomeric 1,2-carborane, as is consistent with the high thermodynamic stability of the closo-1,12-carborane cage. It is considered that cationic, boron-rich compounds 1333 and 15 may find applications as mitochondrial-targeting agents for potential use in BNCT.

### Experimental Details

All reactions were performed at under a dry nitrogen atmosphere. All manipulations were performed using conventional Schlenk techniques.

Distilled water was used for all experiments requiring water. THF, CH₂Cl₂ and CH₃CN were dried prior to use. THF was dried over sodium wire and freshly distilled from benzophenone ketyl before use. Anhydrous CH₂Cl₂ was freshly distilled from CaH₂ before use. All other solvents were used without purification. All precursor chemicals used were commercially available. 1,2-, 1,7- and 1,12-carborane were purchased from Katchem (Czech Republic). All other reagents were available from Aldrich Chemical Co. Phosphorus trichloride was purified by distillation. All other chemicals were used without purification.

All ¹H, ¹³C{¹H}, ¹¹B{¹H} and ³¹P{H}NMR spectra were recorded at 300 K on a Bruker DRX400, DRX200 or DRX300 spectrometer (¹H at 400 MHz, ¹³C{¹H} at 101 MHz, ¹¹B{¹H} at 128 MHz and ³¹_{P{}¹H} at 162 MHz unless otherwise stated). All NMR signals (δ) are reported in ppm. ¹H and ¹³C{1H} spectra in CDCl₃ were referenced to TMS at 0 ppm. ¹H NMR spectra in all other solvents were referenced according to their residual solvent peaks. ¹¹B{¹H} NMR spectra were referenced to external standard BF₃•OEt₂ at 0 ppm. ³¹P{¹H}NMR spectra were referenced to external standard P(OMe)₃ at 140.85 ppm. Commercially available deuterated solvents of 99.5% isotopic purity or higher were used for all spectra. Mass spectra were acquired in an appropriate solvent (flow rate 100 μL/min) on a Finnegan LCQ MS Detector (ESI) or Polaris Q MS Detector (EI). An ESI spray voltage of 5 kV was applied with a heated capillary temperature of 200° C. and a nitrogen sheath gas pressure of 60 psi. Melting points were determined using a Gallenkamp digital melting point apparatus and are uncorrected. Elemental analyses were performed by Chemical and Microanalytical Services Pty. Ltd., Belmont, Victoria or by the Campbell Microanalytical Laboratory, University of Otago, New Zealand.

### (1,12-dicarba-closo-dodecaboranyl)diphenyl(1-bromo-2-(2-(2-ethoxyethoxy)ethoxy)ethyl)phosphonium bromide 9

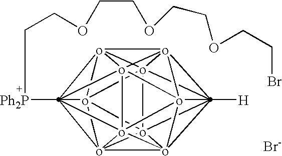

To a stirred solution of 3 (0.975 g, 2.97 mmol) in dimethylformamide (10 mL) was added bis(2-(2-bromoethoxy)ethyl)ether (4.74 g, 14.8 mmol). This was heated at 120° C. for 24 h. The solution was washed with hexane (3×50 mL) and the solvent removed. The residue was dissolved in water (50 mL) and washed with diethyl ether (3×50 mL). To the water layer was added acetone (50 mL) and overnight brown-red impurities precipitated out of solution. The solution was filtered and the solvent removed to give an off-white solid interspersed with red-brown impurity. To this residue was added a small amount of acetone (20 mL) which dissolved the impurity. The insoluble material was filtered off and recrystallised from water. This solid was air-dried and further dried in a dessicator over phosphorus pentoxide to afford the title compound as an off-white solid. (0.45 g, 23%) ¹H NMR (400 MHz, CDCl₃) δ 8.03 (m, 4H), 7.83 (m, 2H), 7.73 (m, 4H), 3.89 (m, 2H), 3.75 (m, 2H), 3.65 (m, 1H), 3.59 (m, 1H), 3.45 (m, 4H), 3.22 (m, 2H), 3.12 (br s, 1H), 3.06 (m, 2H), 2.99 (m, 2H). ³¹P{¹H} NMR (162 MHz, CDCl₃) δ32.01 (s). ¹¹B{¹H} NMR (128 MHz, CDCl₃) −13.1 (br s). m/z (ESI-MS, +ve) 568.4 (M⁺).

### μ-1,12-dicarba-closo-dodecaboranyl-1,12-bis(diphenylmethylphosphonium)iodide 10

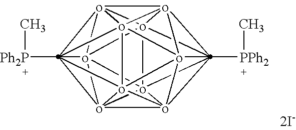

To a solution of 11 (0.723 g, 1.41 mmol) in tetrahydrofuran (5 mL) was added iodomethane (2.00 mL, 32.1 mmol). The mixture was stirred at reflux overnight. The precipitate that formed was filtered off and washed in tetrahydrofuran to yield the title compound a colourless powder (1.12 g, 99%).

¹H NMR (400 MHz, d₇-DMF) δ8.29 (m, 8H), 8.05 (m, 6H), 7.90 (m, 8H), 3.31 (d, ²J_{P,H}=3.28 Hz, 6H). ³¹P{¹H} NMR (162 MHz, d₇-DMF) δ27.70 (s). ¹¹B{¹H} NMR (128 MHz, d₇-DMF) δ-11.3 (br s). Found C 41.28, H 4.36. Calc. for C₂₈H₃₆B₁₀I₂P₂.H₂O: C 41.29, H 4.70%. m/z (ESI-MS, +ve) 271.6 (M²⁺).

### Bis(1-diphenylphosphino-1,12-dicarba-closo-dodecaborane)gold(I) chloride 13

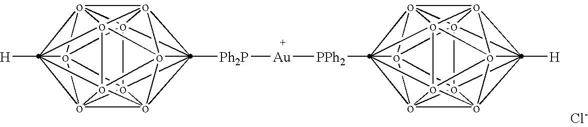

The carboranyl phosphine 3 (0.26 g, 0.79 mmol) and [AuCl(SMe₂)] (0.12 g, 0.40 mmol) were dissolved in dichloromethane (30 mL) and the mixture was stirred overnight at room temperature. The solvent was removed under vacuum to give an off-white solid, which was recrystallized from dichloromethane to afford the title compound as colourless crystals (0.18 g, 52%).

(spectra: JI035_CDCl3_{—}22Aug07)

¹H NMR (400 MHz, CDCl₃) 7.78 (m, 8H, Ph), 7.46 (m, 12H, Ph), 2.81 (br s, 1H, C_{cage}-H). ¹¹B{¹H} NMR (128 MHz, CDCl₃) −11.7 (br s, 10B), −13.4 (br s, 10B). ³¹P{¹H} NMR (162 MHz, CDCl₃) 40.9 (s, 2P).

(spectrum from JI035_CDCl3_{—}01Jul07 exp3)

¹³C{¹H} NMR (101 MHz, CDCl₃) 135.50 (d, ²J_{P,H}=20.63 Hz, Ph), 131.95 (s, Ph), 129.77 (br d, ¹J_{P,H}=22.44 Hz, Ph), 128.81 (d, ³J_{P,H}=11.17 Hz, Ph), 78.40 (br d, ¹J_{P,H}=19.02 Hz, C_{cage}-P), 65.39 (s, C_{cage}-H). m/z (ESI-MS, +ve) 854.442155 (M⁺). Calc. 854.441735 Found C 33.37, H 4.85. Calc. for C₂₆H₃₀B₁₀P₂Au₂Cl₂.(CH₂Cl₂)₂: C 34.02, H 4.38%.

(Note: Not within 0.4%)

### Chloro(1-diphenylphosphino-1,12-dicarba-closo-dodecaborane)gold(I) 12

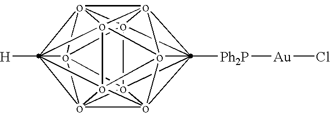

This was prepared using a method similar to that for 13.

Mp>220° C. (decomp.)

(spectra: JI035_Ace_{—}18Sep07)

¹H NMR (400 MHz, d₆-acetone) 8.06 (m, 4H, Ph), 7.67 (m, 6H, Ph), 3.63 (br s, 1H, C_{cage}-H) ¹¹B{¹H} NMR (128 MHz, d₆-acetone) −10.7 (br s, 5B), −12.4 (br s, 5B) ³¹P{¹H} NMR (162 MHz, d₆-acetone) 54.8 (br s) νₘₐₓ (polyethylene)/cm⁻¹ 333 (Au—Cl). Found C 29.95, H 3.92. Calc. for C₁₄H₂₁AuB₁₀ClP: C 29.98, H 3.77%.

### Dichloro-μ-{1,12-bis(diphenylphosphino)-1,12-dicarba-closo-dodecaborane}digold(I) 14

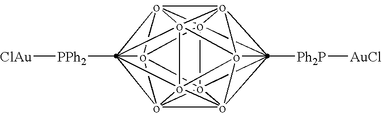

The carboranyl phosphine 11 (0.23 g, 0.44 mmol) and [AuCl(SMe₂)] (0.26 g, 0.90 mmol) were dissolved in dichloromethane (30 mL) and the mixture was stirred overnight at room temperature. The resulting white precipitate was filtered off, washed with dichloromethane, and air-dried. The product was further dried under vacuum over phosphorus pentoxide to afford the title compound as a colourless powder (0.30 g, 70%). Mp >275° C. (decomp.). νₘₐₓ (polyethylene)/cm⁻¹ 334 (Au—Cl). ¹H NMR (400 MHz, CDCl₃) 7.89 (m, 8H, Ph), 7.59 (m, 4H, Ph), 7.51 (m, 8H, Ph). ¹¹B{¹H} NMR (128 MHz, CDCl₃) −11.1 (br s, 10B). ³¹P{¹H} NMR (162 MHz, CDCl₃) 58.5 (s). Found C 31.82, H 2.97. Calc. for C₂₆H₃₀B₁₀P₂Au₂Cl₂: C 31.95, H 3.09%.

### Bis(1,12-bis(diphenylphosphino)-1,12-dicarba-closo-dodecaborane) gold(I) Chloride 15

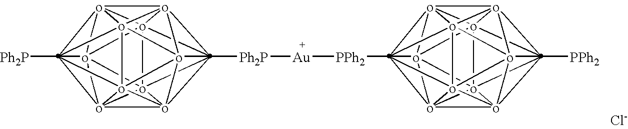

The carboranyl phosphine 11 (0.71 g, 1.38 mmol) and [AuCl(SMe₂)] (0.20 g, 0.69 mmol) were dissolved in dichloromethane (30 mL) and the mixture was stirred overnight at room temperature. The solvent was removed under vacuum to give an off-white solid, which was recrystallized from dichloromethane to afford the title compound as colourless crystals (0.73 g, 85%).

Mp 193-195° C.

¹H NMR (400 MHz, CDCl₃) 7.64 (m, 8H, Ph), 7.39 (m, 12H, Ph). ¹³C{¹H} (101 MHz, CDCl₃) 135.18 (d, ²J_{P,C} 25.8 Hz, Ph), 133.42 (d, ¹J_{P,C} 14.3 Hz, Ph), 130.55 (s, Ph), 128.42 (d, ³J_{P,C} 9.1 Hz, Ph), 80.95 (d, ¹J_{P,C} 60.2 Hz, C_{cage}). ¹¹B{¹H} (128 MHz, CDCl₃) −11.0 (br s, 20B). ³¹P{¹H} (162 MHz, CDCl₃) 28.4 (br s). m/z (ESI-MS, +ve) Found 1222.525347 (M⁺). Calc. for C₅₂H₆₀AuB₂₀P₄ 1222.531948 Found C 45.56, H 4.52. Calc. for C₅₂H₆₀AuB₁₀P₄Cl.(CH₂Cl₂)₂: C 45.44, H 4.52%.

1. A compound comprising a carborane group coupled to a phosphonium group, wherein the phosphonium group is bonded directly to a carbon atom of the carborane group.

2. The compound of claim 1 wherein the phosphonium group is an alkyldiaryl phosphnium group.

3. The compound of claim 2 the alkyldiaryl phosphonium group is diphenylmethyl phosphonium.

4. The compound of claim 1 wherein the carborane group is a closo carborane group, and the compound comprises a counterion to the phosphonium group.

5. The compound of claim 4 wherein the counterion is a halide.

6. The compound of claim 5 wherein the halide is iodide.

7. The compound of claim 1 wherein the carborane group is a dicarba-closo-dodecaborane group.

8. The compound of claim 1 wherein two phosphonium groups are coupled to the carborane group.

9. The compound of claim 1 wherein the carborane group is a nido-carborane group bearing a negative charge, whereby the compound is a zwitterion.

10. The compound of claim 9 wherein the compound is a dicarba-nido-undecaborane.

11. A process for making a compound according to claim 1 comprising alkylating a precursor, said precursor comprising a closo-carborane group coupled to a phosphine group.

12. The process of claim 11 wherein the step of alkylating comprises reacting the precursor compound with an alkylating agent

13. The process of claim 12 wherein the alkyl halide is an alkyl iodide.

14. A process for making the compound of claim 9 comprising the steps of:
a) alkylating a precursor, said precursor comprising a closo-carborane group coupled to a phosphine group, to form a product comprising a closo-carborane group coupled to a phosphonium group; and
b) deboronating the product obtained in step a).

15. The process of claim 14 wherein step b) comprises exposing the product obtained in step a) to a polar solvent.

16. The process of claim 15 wherein the polar solvent comprises a solution of fluoride ion in ethanol.

17. A compound made by the process of any one of claims 11 or 14.

18. A composition comprising a compound according to claim 1 or 9 together with one or more clinically acceptable adjuvants and/or carriers.

19. A method for imaging and/or treating a tumour in a patient comprising the step of administering to said patient a compound according to claim 1 or 9 or a pharmaceutical composition thereof.

20. The method of claim 19 additionally comprising the step of irradiating the tumour with neutrons during or after said step of administering.

21-22. (canceled)

23. A method for imaging or treating a tumor comprising administering a composition of claim 18 to a patient.
